(19) United States
(12) Patent Application Publication
(10) Pub. No.: US 2004/0050385 A1
(43) Pub. Date: Mar. 18, 2004

(54) Inhaler

(76) Inventors: Stanley George Bonney, Ware (GB); Michael Birsba Davies, Ware (GB)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/399,674

(22) PCT Filed: Sep. 26, 2001
(86) PCT No.: PCT/EP01/11096

(30) Foreign Application Priority Data
Oct. 20, 2000 (GB) 0025751.9
Dec. 8, 2000 (GB) 0029979.2

(51) Int. Cl. A61M 15/00

(52) U.S. Cl. 128/203.15

(57) ABSTRACT

The invention provides an inhaler for delivery of a dry powder medicament, the inhaler comprising a breath sensor for sensing the breath of a patient; means for transporting the medicament to a delivery position; and electro-mechanical coupling means for actuating said transport means, wherein said coupling means is directly or indirectly responsive to the breath sensor.

### TECHNICAL FIELD

This invention relates to an inhaler for dispensing dry powder medicaments. In particular, the invention relates to an inhaler that does not require manual actuation by a patient.

### BACKGROUND OF THE INVENTION

Medical dispensers are well known for the dispensing of various kinds of medicament. Inhalation devices, such as metered dose inhalers (MDI) and dry powder inhalers are known for the delivery of medicament for the treatment of respiratory disorders such as asthma and chronic inflammatory pulmonary disease.

There are a number of different dry powder inhalers presently available. In one instance, the drug is encapsulated in hard gelatine and the inhaler comprises a device for perforating a capsule prior to the patient inhaling the contents. After the patient manually activates the opening of the capsule, a cloud of dry particles is directed into the nose or mouth of the patient usually by a channelling device such as a cylinder or open-ended cone. Concurrently with the release of the capsule contents, the patient inhales the drug particles into the lungs or nasal cavity. The vacuum created by the patient on inhalation is intended to empty the capsule contents.

The inhaler exemplified in EP-A-467172 accommodates a blister pack wherein each blister retains a dose of medicament in dry powder form. When a blister is positioned for dosing, a mechanism within the inhaler punctures the blister, releasing the contents for inhalation by the user as described supra. U.S. Pat. No. 4,805,811 discloses a dry powder inhaler comprising a dry powder reservoir from which a dosing plate having a number of dosing “cups” is filled from the reservoir prior to inhalation. As with the examples described supra, this device requires manual metering and/or releasing of a metered dose prior to inhalation.

It may be understood that effective delivery of medicament to the patient using an inhalation device as described is to an extent dependent on the patients ability to manually actuate the device (e.g. puncturing of a capsule) and to coordinate the actuation thereof with the taking of a sufficiently strong inward breath, For some patients, particularly young children, the elderly and the arthritic, manual actuation of the device can present difficulties. Other patients find it difficult to co-ordinate the taking of a reliable inward breath with actuation of the device. Both of these sets of patients run the risk that they do not receive the appropriate dose of medicament.

U.S. Pat. No. 5,239,992 discloses a loose powder inhaler wherein the vacuum created on inhalation by the user drives a dosing piston to measure and liberate a dose concurrent with inhalation of the drug However, this device is reliant on the patient being able to draw sufficient breath to create the necessary vacuum and therefore does not alleviate the problems discussed supra.

The Applicants have now developed a dry powder inhaler that does not require manual actuation by the patient.

### SUMMARY OF THE INVENTION

Accordingly, in one aspect, the invention provides an inhaler device for delivery of a metered dose of dry powder medicament, the inhaler comprising a breath sensor for sensing the breath of a patient; means for transporting the medicament dose to a delivery position; and electromechanical coupling means for actuating said transport means to transport the medicament dose to said delivery position, wherein said coupling means is directly or indirectly responsive to the breath s ns r.

As used herein the term “transport means” refers to means for transferring the metered volume of medicament from a non-delivery, metering position, to a position where the drug is ready for delivery to the patient.

Transport of the medicament dose to a delivery position is wholly dependent on the actuation of the breath sensor by the patient's breath. Accordingly, the medicament is protected from unintentional manual actuation of the dispenser whereby the dose may be lost or exposed to the environment.

In one embodiment, the medicament is pre-metered prior to actuation of the inhaler by the patient, for example, the medicament is pre-metered in capsules, strip or tape form.

Preferably, the inhaler further comprises a reservoir for said dry powder and a meter for metering an amount (erg. by volume or by weight) of dry powder from said reservoir.

Preferably, the breath sensor actuates said meter.

In one embodiment, the coupling means is responsive to said meter. In this embodiment, the patient's breath would activate the metering mechanism which subsequently actuate the transport means.

Preferably, the inhaler further comprises release means. The release means may be actuable by the coupling means and/or the meter and/or the transport means.

As used herein, the term “release means” refers to the means for the making available of the dose for release to the patient, for example, the actual dispensing (whether passive or active) to the patient. The release may be active in the sense that medicament is actively dispensed from the container, or the release may be passive in the sense that medicament is merely made available for release when the release means is actuated.

Typically, the breath sensor and/or the meter and/or the transport means actuate the release means immediately after, or concurrent with, the actuation of the meter.

In this embodiment, the invention ensures that only after a dose has been metered from the dry powder reservoir can the medicament be made available for inhalation by the patient. Accordingly, the metered dose does not remain waiting in a metering chamber or delivery unit or release chamber for any length of time and therefore there is substantially reduced or alleviated the chance of deposition or sticking of the medicament onto the walls of the device, or the chance of moisture ingress or contamination from the external environment.

A reset mechanism may be provided for resetting the transport and optionally the release means after actuation thereof. The reset means may for example, comprise a spring, motor, or other mechanical arrangement, and/or an electronic arrangement.

The release means may comprise (i) a passive and/or (ii) an active dose-release mechanism.

Typically, the release means is linked to the transport means and/or the meter and/or the breath sensor such that the release means is actuated immediately after metering and transport of a dose.

In one embodiment, the release means is passive and comprises making the metered dose available to the patient for inhalation thereby.

In another embodiment, the release means is active and comprises means to propel pressurised gas in the direction of patient inhalation. In this embodiment, the patient receives a positive signal that the dose has been dispensed which may add to patient confidence. An active release means may also increase the efficacy of delivery of the medicament, for example, the drug may be released in a more focussed plume or cloud towards the back of the inhaler's nose or throat.

Preferably, the gas-propelling means provides at least one pulse of gas on actuation.

The gas-propelling means may provide one pulse of gas for each dose dispensed.

The gas may be air or an inert gas.

Preferably, the meter is linked to the breath sensor such that the breath sensor actuates the meter immediately prior to, or concurrent with, actuation of the release means.

Metering of the dry powder medicament immediately prior to inhalation has a number of advantages. Firstly, the medicament has no time to absorb moisture from its environment outside the dry powder reservoir. Also, the problem of medicament adhesion or sticking to the metering mechanism is alleviated or substantially reduced.

Typically, the meter comprises a volume and/or time and/or surface area regulated mechanism.

In one embodiment, metering of medicament dose may be achievable by pulsing electrical current flow through the meter for a selected dispensing time.

For example, the meter may comprise a valve (for example, a linear or rotary valve) and/or a piston and/or a load cell. In another aspect, the meter may comprise a plunger, such as might exist in a syringe. Embodiments including multiple plungers and multiple syringe chambers are also envisaged.

Preferably, the meter comprises at least one metering chamber. In one embodiment, on actuation of the meter, the or each metering chamber moves into fluid communication with the reservoir.

In one embodiment, the meter and the reservoir are relatively rotatable with respect to each other about a common central axis. Preferably, the or each metering chamber is adapted to be in fluid communication selectively with the reservoir or with the patient.

The or each metering chamber may have a variable volume. Alternatively, the or each metering chamber may have a fixed volume which is variable by insertion of a plunger or piston. The or each metering chamber may be formed from expandable material and/or have a telescopic or concertina arrangement.

In one embodiment, the inhaler further comprises a gas permeable dry powder retaining means below the or each metering chamber. The retaining means may be made from a gas-permeable filter, a mesh screen, a porous material or a perforated chamber element.

In another embodiment, the inhaler additionally comprises climate control means. Preferably, the climate control means is actuable by the coupling means and/or the meter and/or the transport means and/or the release means.

The climate control means may comprise means to (i) reduce moisture increase in the inhaler; and/or (ii) maintain ambient temperature; and/or (iii) dry the meter prior to actuation of the inhaler.

The climate control means may comprise a desiccant and/or a heater.

The heater may dry the meter prior to metering of the dose and/or immediately after the dose is dispensed.

The climate control means may comprise a temperature and/or a moisture sensor.

The coupling means may comprise a spring and/or a lever. Alternatively or in addition, the coupling means may comprise a solenoid.

In one embodiment, the coupling means is reversibly deformable in response to heating thereof or application of a magnetic field thereto.

The inhaler may additionally comprise a reset coupling which is reversibly deformable in response to heating thereof or application of a magnetic field thereto.

Preferably, heating is achievable by electric current flow through the coupling or reset coupling.

Preferably, the coupling or reset coupling comprises a wire, strip, coil or tube.

Arrangements comprising multiple strips, wires, coils, or tubes are also envisaged. The multiple strips, wires, coils, or tubes may be arranged in any suitable fashion including parallel or series arrangements and bundle arrangements.

In one particular aspect, the coupling or reset coupling comprises one or more wires which contract in response to heating or application of a magnetic field thereto.

Preferably, the degree of contraction of the coupling is from 2% to 8%.

In embodiments, the coupling comprises an alloy which undergoes a phase transition on heating (shape memory alloys). Certain shape memory alloys also undergo a change in shape on recooling. Such two way shape memory alloys are also envisaged for use herein.

In one embodiment, the shape memory alloy is preferably a nickel-titanium alloy such as a nickel-titanium alloy comprising from 5% to 95%, preferably from 20% to 80%, nickel by weight and from 95% to 5%, preferably from 80% to 20%, titanium by weight. By nickel-titanium alloy it is meant an alloy comprised essentially of nickel and titanium, although other elements such as Cu and Nb may be present in small (e.g. trace) amounts.

In other embodiments, the shape memory alloy is preferably a copper-aluminium-nickel alloy or a copper-zinc-aluminium alloy. Trace amounts of other elements may also be present.

In further embodiments, the coupling comprises an alloy which undergoes a phase transition on application of a magnetic field thereto (magnetic shape memory alloys). These materials are generally intermetallic, ferromagnetic alloys that exhibit twin variants in the martensitic, or low-temperature, phase of the material. Suitable magnetic shape memory alloys are for example, described in U.S. Pat. No. 5,958,154.

In one embodiment, the magnetic shape memory alloy exhibits an austenitic crystal structure above a characteristic phase transformation temperature and also exhibits a martensitic twinned crystal structure below the phase transformation temperature. The alloy has a magnetocrystalline anisotropy energy that is sufficient to enable motion of twin boundaries of the martensitic twinned crystal structure in response to application of a magnetic field to the martensitic twinned crystal structure.

Where a magnetic shape memory alloy is employed the inhaler preferably includes a magnetic field source disposed with respect to the coupling in an orientation that applies to the coupling a magnetic actuation field in a direction that is substantially parallel with a selected twin boundary direction of the martensitic twinned crystal structure of the coupling material.

Alternatively, the inhaler preferably includes a magnetic bias field source disposed with respect to the coupling in an orientation that applies a magnetic bias field to the coupling, and a magnetic actuation field source disposed with respect to the coupling in an orientation that applies a magnetic actuation field to the coupling material in a direction that is substantially perpendicular to the orientation of the applied magnetic bias field.

A preferred magnetic shape memory alloy is the actuator material comprising an alloy composition defined as Ni_{65-x-y}Mn₂₀+xGa₁₅+y, where x is between 3 atomic % and 15 atomic % and y is between 3 atomic % and 12 atomic %. Preferably, the actuator material comprises an alloy composition defined as Ni_{65-x-y}Mn₂₀+xGa₁₅+y, where x is between 6 atomic % and 10 atomic % and y is between 5 atomic % and 9 atomic %; or where x is between 12 atomic % and 15 atomic % and y is between 3 atomic % and 6 atomic %; or where x is between 10 atomic % and 14 atomic % and y is between 3 atomic % and 6 atomic %; or where x is between 7 atomic % and 11 atomic % and y is between 3 atomic % and 7 atomic %. In a particularly preferred aspect, the alloy is Ni₅₀Mn₂₅Ga₂₅.

Another preferred magnetic shape memory alloy is the alloy having the composition (NiₐFe_{b}Co_{c})_{65-x-y}(Mn_{d}FeₑCo_{f})₂₀+x(Ga_{g}SiₕAlᵢ)₁₅+y, where x is between 3 atomic % and 15 atomic % and y is between 3 atomic % and 12 atomic %, and where a+b+c=1, where d+e+f=1, and g+h+i=1.

In preferred aspects, b is between zero and 0.6, c is between zero and 0.6, and e, f, h and i are each zero; or b and c are each zero, e is between zero and 0.6, f is between zero and 0.6, and h and i are each zero; or b, c, e and f are each zero, h is between zero and 0.5, and i is between zero and 0.5.

Preferably, the one or more wires have a diameter from 30 to 400 micrometers, preferably from 50 to 150 micrometers.

Preferably, the coupling comprises from two to twelve, preferably six to ten wires which contract in response to heating or application of a magnetic field thereto. The wires may be arranged in any suitable fashion including parallel or series arrangements and bundle arrangements.

In another aspect, the coupling comprises a strip which comprises multiple layers of different metals. Suitable strips typically comprise a plurality of layers of material, each material having a different coefficient of thermal expansion.

Preferred examples of strips include those comprising multiple layers of different metals (e.g. bimetallic strips) and strips comprising at least one piezoelectric material. Suitable piezoelectric materials include piezoelectric ceramics, such as compounds of lead zirconate and lead titanate, and piezoelectric crystals which are generally polycrystalline ferroelectric materials with the perovskite structure.

In one aspect, the coupling is deformable in response to heating arising from electrical current flow in the range from 0.01A to 100A, preferably from 0.1A to 5A. Alternatively, the coupling is deformable in response to heating arising from the application of an electrical voltage, particularly where the coupling comprises a piezoelectric material.

In another aspect, the coupling is deformable in response to a magnetic field of from 0.01 to 100 Tesla. The magnetic field may for example, be produced by a permanent magnet or by an electromagnet.

The deformation of the coupling (e.g. by electrical current flow therethrough) may be responsive to the detection of the inward breath of a patient. Alternatively, deformation of the coupling (e.g. by electrical current flow therethrough) may be responsive to a trigger coupled to any point in the breathing pattern of the patient, such as the end of the outward breath.

As used herein the term breath sensor encompasses any suitable means for monitoring, measuring, tracking or indicating the breath of a patient and may comprise one or more sensors.

In one aspect, the sensor comprises a breath-movable element which is movable in response to the breath of a patient. Preferably, the breath-movable element is selected from the group consisting of a vane, a sail, a piston, a diaphragm and an impeller.

Movement of the breath-movable element may be detectable by any suitable technique for detecting movement. Suitable techniques include optical detectors, magnetic detectors or detectors using detection of capacitative effects.

Optical detectors may be used to detect movement of the breath-movable element by providing the element with a patterned outer surface, for example strips in a barcode type arrangement, and locating the optical detector so that it points towards the patterned surface. Movement of the breath-movable element alters the amount of the light source which reflects back onto the optical detector as the beam passes over the patterned surface. The strips may be arranged so that the direction of movement of the element can be detected.

Magnetic detectors may be used to detect the movement of breath-movable element by the use of a magnetic switch device. A reader is located on the dispenser and magnetic material embedded within the breath-movable element (or vice-versa). Movement of the breath-movable element results in a change of the magnetic field experienced by the reader. Alternatively, a Hall effect device can be used whereby a semiconductor measures the strength of the magnetic field of the magnetic material on the breath-movable element.

Detection of capacitative effects may be used to detect movement of the breath-movable element by adding a conductive part to the element and also to a second fixed part of the dispenser. Movement of the breath-movable element results in a change in capacitance which can be measured.

In another aspect, the sensor comprises a pressure sensor for sensing the pressure profile associated with the breath of a patient. A pressure transducer is an example of a suitable pressure sensor.

In another aspect, the sensor comprises an airflow sensor for sensing the airflow profile associated with the breath of a patient.

In another aspect, the sensor comprises a temperature sensor for sensing the temperature profile associated with the breath of a patient.

In another aspect, the sensor comprises a moisture sensor for sensing the moisture profile associated with the breath of a patient.

In another aspect, the sensor comprises a gas sensor for sensing the chemical profile, for example, the oxygen or carbon dioxide profile associated with the breath of a patient.

Preferably, the sensor is connectable to an electronic information processor. The connection may be direct or via any suitable mechanical or electronic transfer means.

Preferably, the electronic information processor actuates the meter at a predetermined trigger point in the breath cycle.

Preferably, the electronic information processor includes an active memory for storing information about the breath cycle.

Suitably, the electronic information processor includes a predictive algorithm or look-up table for predicting the optimum trigger point. For example, a real-time analysis of the patient waveform may be made and the optimum trigger point derived by reference to that analysed waveform.

Suitably, the electronic information processor includes a second predictive algorithm or look-up table for predicting the optimum amount of medicament to release. Suitably, the electronic information processor includes a dose memory for storing information about earlier delivered doses and reference is made to the dose memory in predicting the optimum amount of medicament to release.

Preferably, the inhaler additionally comprises a display for displaying information about the optimum amount of medicament to release.

Preferably, the inhaler according additionally comprises a selector for selecting the amount of medicament to release.

In one aspect, the selector is manually operable.

Alternatively or in addition, the selector is operable in response to a signal from the electronic information processor.

Preferably, the selector comprises a timing mechanism for varying the time interval of actuation of the dose-metering and/or dose-release mechanism.

The selector may comprise a multiple-fire mechanism for multiple actuation of the inhaler wherein each actuation releases a portion of the optimum amount of medicament.

Preferably, the inhaler additionally comprises an electrical energy source. In one aspect, the electrical energy source comprises a voltaic cell or battery of voltaic cells which may be rechargeable. In another aspect, the electrical energy source comprises a photovoltaic cell or battery of photovoltaic cells. The additional energy source may be mechanically-generated, for example, the energy source may comprise a biasable resilient member, e.g. a spring. Therefore, the electrical energy source may comprise a converter for converting mechanical energy into electrical energy.

The energy source may comprise a source of compressed fluid, preferably compressed gas, or a chemical energy store, preferably a chemical propellant or ignition mixture. Other sources may include physical explosives such as liquefied or solidified gas in a canister which burst when heated or exposed to the atmosphere.

Any electrical circuit may incorporate voltage amplification means for generating a higher voltage than that supplied by the voltaic cell or battery of voltaic cells, for example a step-up or inverting switching circuit or a dc-dc converter incorporating an oscillator, transformer and rectifier.

The electrical circuit may incorporate one or more energy storage components such as capacitors or inductors in order to supply a high enough instantaneous current to raise the temperature of the strips or wires at the required rate to the required temperature.

The input to the electrical circuit may be connected to the electrical energy source by means of a mechanical, electromechanical or electronic switching component.

The output of the electrical circuit may be connected to the strips or wires or to an electromagnet by means of a mechanical, electromechanical or electronic switching component or by a component allowing the output current to be controlled in a linear or digital (e.g. pulse width modulated) manner.

The strip or wire components may be powered from the battery using a switching component without additional power supply circuitry.

Suitably, the inhaler additionally comprises a controller for controlling the amount of electrical current flow through the coupling or to an electromagnet.

Suitably, the inhaler additionally comprises a timer for controlling the duration of electrical current flow through the coupling or to an electromagnet.

Suitably, the inhaler additionally comprises a local electrical store such as a capacitor or inductor.

Suitably, the inhaler is provided with a manual override to enable actuation of the device in the event of loss of electrical power. For example in the event of an emergency or system failure.

Preferably, the inhaler includes a safety mechanism to prevent unintended multiple actuations of the device. The patient is thereby protected from inadvertently receiving multiple doses of medicament in a situation where they take a number of short rapid breaths. More preferably, the safety mechanism imposes a time delay between successive actuations of the device. The time delay is typically in the order of from three to thirty seconds.

Preferably the inhaler comprises an actuation or dose counter for counting the number of actuations of the meter or dose-release means or releases of dose therefrom. More preferably, counting will occur even if the metering and/or release means is manually actuated, that is, the actuation counter is independent of the coupling between the breath sensor and the dose-dispensing means.

The actuation counter may be mechanical or electronic.

Suitably, the inhaler is provided with child-resistance features to prevent undesirable actuation thereof by a young child.

The inhaler of the invention is suitable for dispensing medicament, particularly for the treatment of respiratory disorders such as asthma and chronic obstructive pulmonary disease (COPD).

Appropriate medicaments may thus be selected from, for example, analgesics, e.g., codeine, dihydromorphine, ergotamine, fentanyl or morphine; anginal preparations, e.g., diltiazem; antiallergics, e.g., cromoglycate, ketotifen or nedocromil; antiinfectives e.g., cephalosporins, penicillins, streptomycin, sulphonamides, tetracyclines and pentamidine; antihistamines, e.g., methapyrilene; anti-inflammatories, e.g., beclomethasone dipropionate, fluticasone propionate, flunisolide, budesonide, rofleponide, mometasone furoate or triamcinolone acetonide; antitussives, e.g., noscapine; bronchodilators, e.g., albuterol, salmeterol, ephedrine, adrenaline, fenoterol, formoterol, isoprenaline, metaproterenol, phenylephrine, phenylpropanolamine, pirbuterol, reproterol, rimiterol, terbutaline, isoetharine, tulobuterol, or (−)-4-amino-3,5-dichloro-α-[[[6-[2-(2-pyridinyl)ethoxy]hexyl]methyl]benzenemethanol; diuretics, e.g., amiloride; anticholinergics, e.g., ipratropium, tiotropium, atropine or oxitropium; hormones, e.g., cortisone, hydrocortisone or prednisolone; xanthines, e.g., aminophylline, choline theophyllinate, lysine theophyllinate or theophylline; therapeutic proteins and peptides, e.g., insulin or glucagon. It will be clear to a person skilled in the art that, where appropriate, the medicaments may be used in the form of salts, (e.g., as alkali metal or amine salts or as acid addition salts) or as esters (e.g., lower alkyl esters) or as solvates (e.g., hydrates) to optimise the activity and/or stability of the medicament.

Medicaments can also be delivered in combinations. Preferred formulations containing combinations of active ingredients contain salbutamol (e.g., as the free base or the sulphate salt) or salmeterol (e.g., as the xinafoate salt) in combination with an antiinflammatory steroid such as a beclomethasone ester (e.g., the dipropionate) or a fluticasone ester (e.g., the propionate). A particularly preferred combination comprises salmeterol xinafoate salt and fluticasone propionate.

Preferred medicaments are selected from albuterol, salmeterol, fluticasone propionate and beclomethasone dipropionate and salts or solvates thereof, e.g., the sulphate of albuterol and the xinafoate of salmeterol, and any mixtures thereof. Alternatively, the dispenser may be employed for dispensing vaccine.

Indeed, it is envisioned in accordance with this invention that any suitable diagnostic, prophylactic or therapeutic agent can used with the inhaler herein. Generally, drug particles suitable for delivery to the bronchial or alveolar region of the lung have an aerodynamic diameter of less than 10 micrometers. Other sized particles may be used if delivery to other portions of the respiratory tract is desired, such as the nasal cavity, mouth or throat. The medicament may be a pure drug, but more appropriately, it is preferred that powder comprise a drug mixed with a bulking agent (excipient), for example, lactose.

Additional powders may be engineered with particular densities, size ranges, or characteristics. Particles may comprise active agents, surfactants, wall forming materials, or other components considered desirable by those of ordinary skill.

Blends of bulking agents and drugs are typically formulated to allow the precise metering and dispersion on the powder into doses. A standard blend, for example, contains 13000 micrograms lactose mixed with 50 micrograms drug, yielding an excipient to drug ratio of 260:1. Because the present invention can meter and dispense such blends more accurately and effectively, dosage blends with excipient to drug ratios of 60:1, and potentially 2:1, may be used. At very low blend levels, however, the drug dose reproducibility becomes more variable.

Typically, the dry powder medicament includes a pharmaceutical excipient in dry powder form.

In one embodiment, the density of the dry powder medicament particles is reduced relative to standard dry powder medicament.

In another embodiment, the dry powder medicament particles are aerodynamically shaped to improve medicament delivery to the patient.

According to another aspect of the present invention there is provided an actuator for a dry powder medicament container having a meter, the actuator comprising a dispenser seat for receipt of the meter, a breath sensor, and transport means, wherein the transport means is electro-mechanically actuable by the breath sensor and/or the meter.

Preferably, the actuator further comprises release means.

Typically, the breath sensor is linkable to the transport means and/or release means via coupling means.

The coupling means may be reversibly deformable In response to heating thereof or application of a magnetic field thereto.

In another aspect, the invention provides a dry powder medicament container having transport and optionally dose-release means for use in the inhaler or the actuator as described hereinabove.

In still a further aspect, the invention provides a kit of parts comprising an inhaler as described hereinabove in the form of a cartridge; and a housing shaped for receipt of the cartridge.

In yet another aspect, the invention provides a method for the delivery of a metered dose of dry powder medicament to a patient comprising:

(i) sensing the breath of a patient by use of a breath sensor;

(ii) transporting the medicament dose to a delivery position by use of transport means; and optionally

(iv) releasing the medicament dose for inhalation by the patient by the use of dose-release means,

wherein electro-mechanical coupling means actuate said transport means and optionally said release means, and said coupling means is directly or indirectly responsive to the breath sensor.

In one embodiment, the medicament is pre-metered prior to actuation of the inhaler by the patient.

In another embodiment, the method includes metering a volume of dry powder from a medicament reservoir after sensing the breath of a patient and prior to transporting the medicament to a delivery position.

Preferably, the metering step is actuable by the breath sensor.

### BRIEF DESCRIPTION OF THE DRAWINGS

The invention will now be described further with reference to the accompanying figures in which:

FIG. 1 shows a typical patient inhalation profile of airflow (litres per minute) against time (seconds) as a patient inhales using a medicament dispenser according to the invention;

FIG. 2 shows a flow diagram of the sequence of events during the dispensing of a dose of medicament to a patient, wherein the inhaler includes a heater to dry the meter apparatus according to one aspect of the invention;

FIG. 3a shows a medicament dispenser according to one aspect of the invention having a medicament reservoir in a vertical orientation;

FIG. 3b shows a medicament reservoir according to one aspect of the invention having a medicament reservoir in a lateral orientation;

FIG. 4a shows a mechanism whereby a DC motor transforms electrical energy into rotary and then linear motion of a dose plate transport means;

FIG. 4b shows a mechanism whereby a DC motor transforms electrical energy into rotary motion of a disk transport means;

FIG. 4c shows a mechanism whereby a DC motor transforms electrical energy into rotary motion of a drum transport means;

FIG. 5 shows a medicament dispenser according to another aspect of the invention having a medicament carrier containing a plurality of individual doses; and

FIG. 6 shows a medicament dispenser as shown in FIG. 3a and an associated system diagram linking the transport means to an electromechanical coupling according to one aspect of the invention.

### DETAILED DESCRIPTION OF THE DRAWINGS

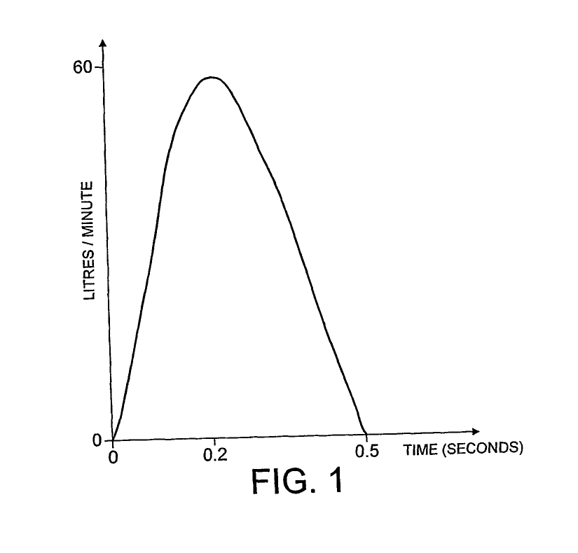

Referring now to the figures, a typical inhalation profile of an adult patient is illustrated in FIG. 1. In this example, the maximum airflow is 60 litres min⁻¹, approximately 200 ms after the breath is initiated. Optimally, a medicament dispenser responsive to a breath sensor should complete the dispensing cycle and make available the medicament for inhalation before the end of the patient's breath cycle.

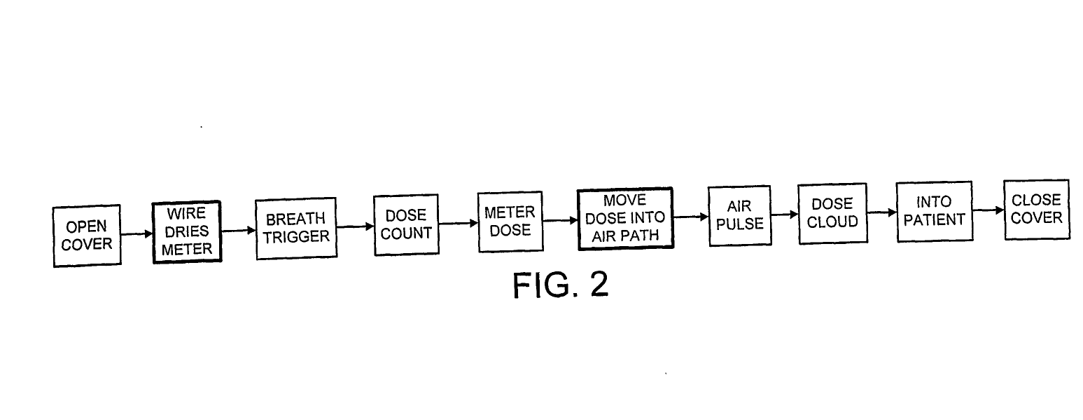

FIG. 2 illustrates the sequence of events during the use of a medicament dispenser in the form of a dry powder inhaler. If the inhaler comprises a protective cover, the cover is removed to expose a mouthpiece. Opening the protective cover activates a heating element to dry a metering pocket in the inhaler and thus alleviate any problems associated with condensation or general environmental moisture. After the patient starts to inhale, a medicament dose is metered from a medicament reservoir. The intake of breath activates a breath sensor and a flow sensor which at a threshold airflow value actuates the transport of the metered dose from a non-dispensing position to a delivery position wherein the medicament is ready for inhalation by the patient. Aerosolisation means in the form of an air pulse generator produces a dose cloud directed towards the patient through the mouthpiece so that the patient may sense the dose entering the mouth and thus the target airways. Notably, the patient has not had to provide any manual intervention throughout the entire metering and dispensing sequence.

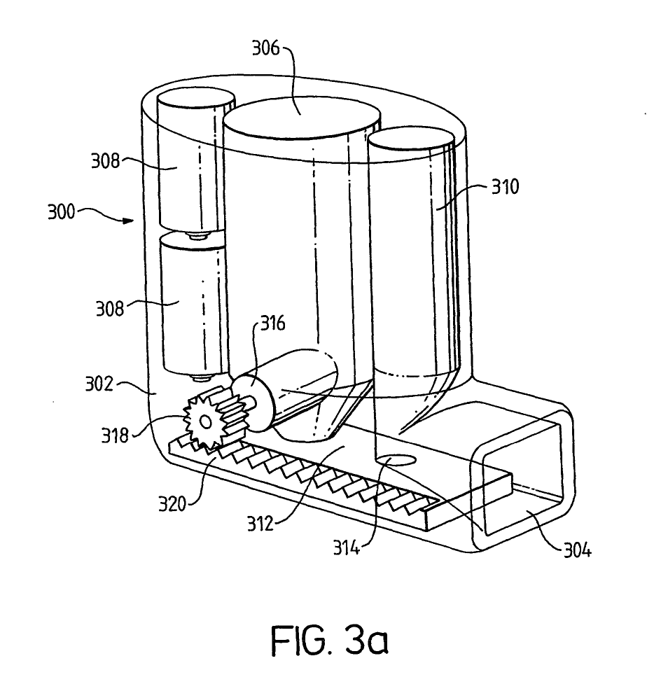

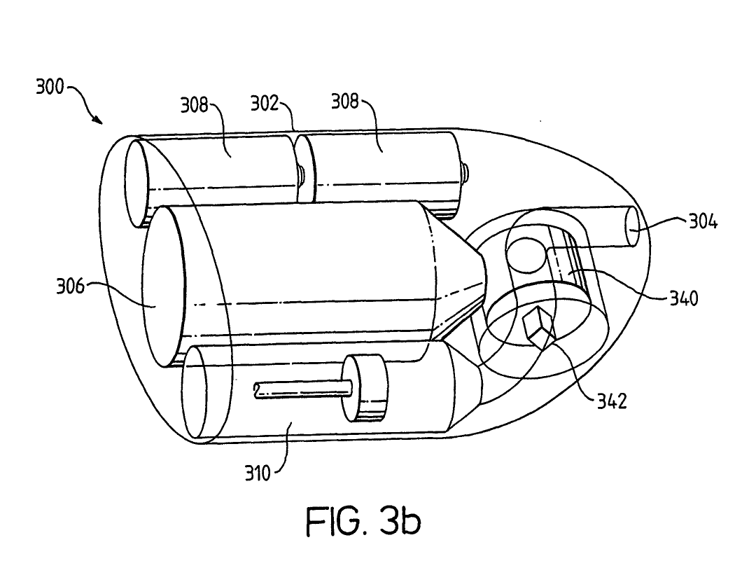

Examples of two different configurations of medicament dispensers according to the invention are illustrated in FIG. 3a and FIG. 3b. FIG. 3a shows an inhaler 300 having a body 302 and a mouthpiece 304. Housed within the body 302 is a medicament reservoir 306 containing dry powdered medicament. The reservoir 306 is in an upright position alongside a power source in the form of two batteries 308 and an air pulse syringe 310. Slidably movable underneath the medicament reservoir 306 is a sliding dose plate 312 (illustrated in the delivery position). A dose pocket 314 is shown exposed to the mouthpiece outlet 304 such that when the dose pocket is full of medicament, the dose is freely available for delivery to the patient. Operation of the inhaler is as follows: as the patient inhales through the mouthpiece 304 a breath sensor comprising a flow sensor (not shown) actuates a rotary DC motor 316 at a predetermined airflow threshold. The DC motor 316 drives a rotary gear wheel 318 which moves the rack 320 on the sliding plate 312 in a linear (back and forth) direction. The dose pocket 314 is transported from directly underneath the reservoir 306 from which a dose of medicament has been transferred to the pocket, to the delivery position as shown in FIG. 3a. The patient can thus inhale the dose through the mouthpiece. After inhalation a reset mechanism (not shown) returns the dose plate 312 to the non-delivery position. Although the figure shows only one dose pocket 314, it can be envisaged that there may be a plurality of dose pockets and further, actuation of the inhaler may result in the metering and transporting of dose on more than one pocket to the patient. In this case the inhaler will further comprise a dose controlling means. Thus the invention is relevant to unit dose inhalers, single-dose inhalers, and multi-dose inhalers.

The inhaler may also comprise a heater (not shown), in the form of a wire, to dry the dose plate 312 prior to metering a dose of medicament. This has the advantage of removing any moisture from the dose plate that might adversely affect the metering of a dose. The heater may be triggered either immediately prior to metering a dose, immediately post metering a dose, or immediately after a dose has been dispensed.

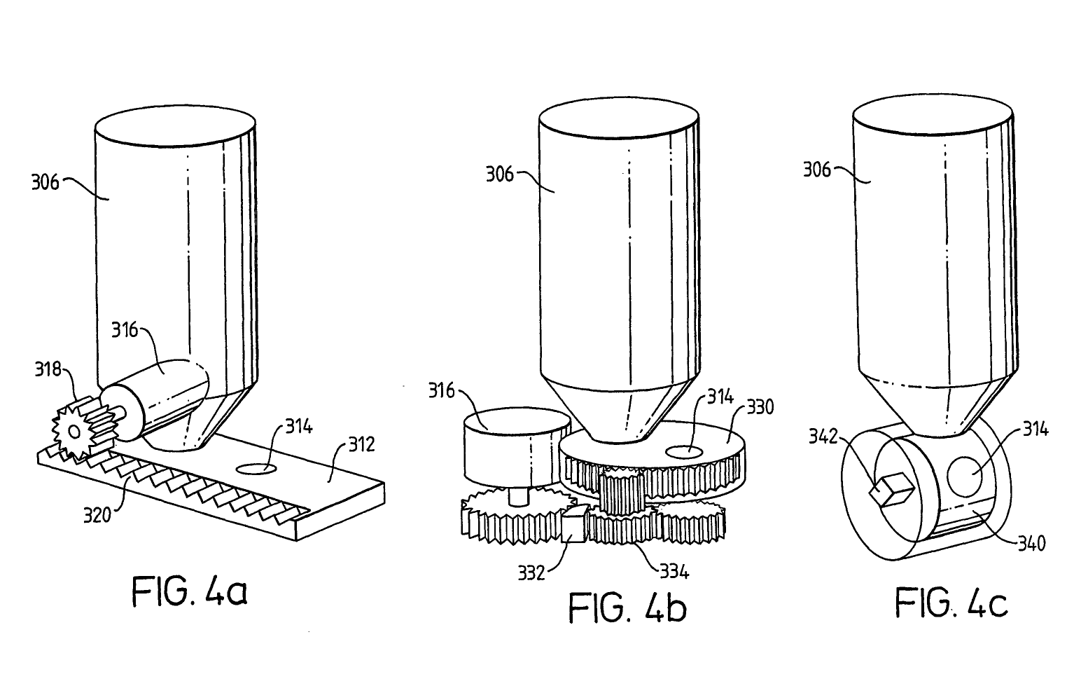

The transporting means may thus comprise a DC motor 316 for generating rotary motion and means for transforming it to linear movement (for example gearing means—this is embodied in FIG. 4a). Alternative methods of creating rotary motion include a piezo-electric motor e.g. an ultrasonic motor. Rotary motion can be transformed to linear motion of a sliding plate as in FIG. 3a and FIG. 4a or it may be transformed into the rotary motion of a disc dose plate 330 as shown in FIG. 4b. In this case, at least one pocket can be rotated from a drying position to a non-dispensing position (e.g. a metering position) to a delivery position (as shown in FIG. 4b). A gearbox 332 is illustrated in FIG. 4b which may be included to change the ratio of gearing between the various gear wheels, e.g. 334.

Linear motion of a sliding dose plate may also be generated using nickel-titanium shape memory alloy wires (SMA wires) which contract on application of an electric current.

FIG. 3b shows an alternative internal arrangement in a medicament inhaler according to the invention. In this example, the medicament reservoir 306 is lying in the same plane as the mouthpiece 304. The dose plate takes the form of a drum 340, the dose pocket(s) being arranged around the circumference of the drum 340. A DC motor 342 is positioned inside the drum 340. Actuation of the inhaler switches on the DC motor 342 which drives the rotary movement of the drum 340. Once again, an air pulse syringe 310 is actuated once the dosing drum 340 is in the delivery position to disperse the dose of medicament in a cloud.

FIG. 4c illustrates the mechanism using a rotating dosing drum 340. An ultrasonic motor may be used in this example as an alternative to a DC motor. The motor 342 may have a number of stepped positions for either drying the pocket, metering a dose or delivering the dose.

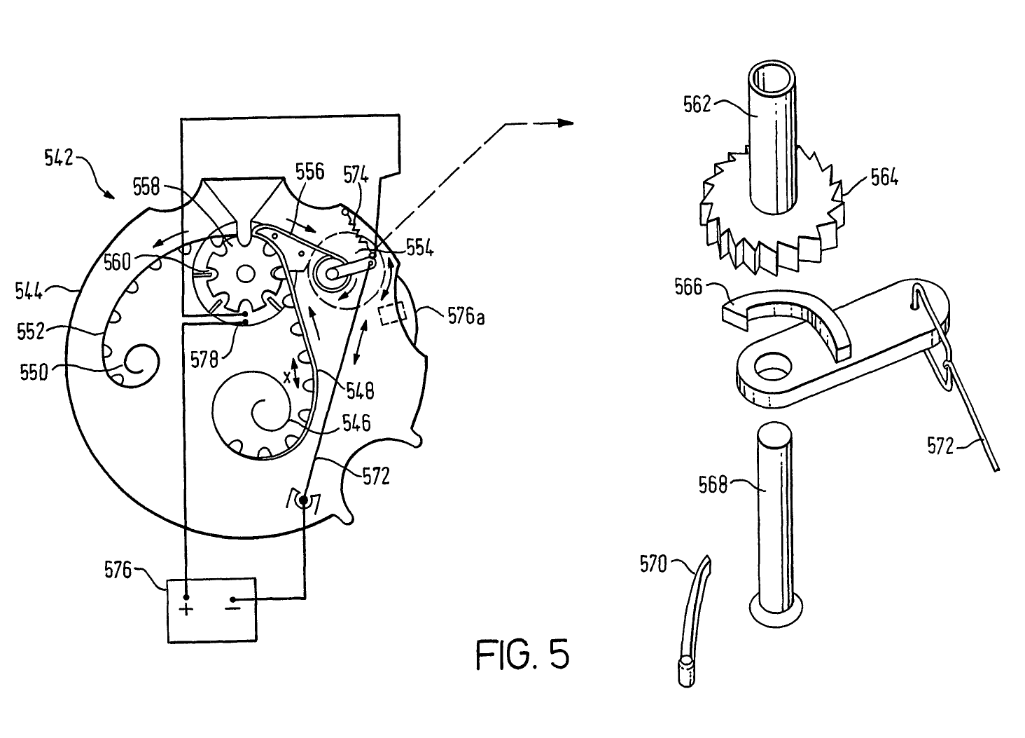

A similar mechanism may be used for the inhaler arrangement illustrated in FIG. 5 (see infra). FIG. 5 illustrates a cross-sectional view through an inhalation device for use with a medicament pack in which dry powder medicament is defined between two sides (a base sheet and a lid sheet) of a peelable strip.

The inhaler 542 has a body 544 defining three storage chambers: one chamber 546 is for housing the strip 548 and from which it is dispensed, one chamber 550 is for receiving the used portion of the base sheet 552, and one chamber 554 is for receiving the used portion of the lid sheet 556.

There is also a chamber for housing an index wheel 558 which has a plurality of grooves 560 spaced at a pitch equal to the distance (x) between the centre lines of adjacent drug pockets.

The transport means comprises means to rotate the index wheel 558 and a lid spool 562 for collecting the lid sheet 556 after drug is dispensed (see exploded view). The lid spool 562 is mounted on a ratchet wheel 564 the teeth of which are engaged by a flexible driving pawl 566 and mounted on a fixed spindle 568. In order to ensure that the ratchet wheel 564 moves only in one direction, there is a flexible ratchet non-return leg 570.

The transport means comprises a transport coupling which takes the form of an SMA wire assembly. A SMA wire 572 is pivotally linked to the driving pawl assembly 576 which is biased to lock the ratchet wheel 564 in position by a return spring 574. A power supply 576 in the form of a battery is linked to the SMA wire 572 such that on actuation of the transport means via a breath sensor 576a an electrical current passes through the SMA wire 572 causing it to heat and contract. As the SMA wire 572 contracts, the driving pawl 566 releases the ratchet wheel 564 to rotate by one or more discrete doses on the medicament strip 548. When the index wheel 558 reaches the following pocket position a contact switch 578 stops the current to the wire 572 which cools, expands and locks the ratchet wheel 564 in position once again.

In alternative embodiments, a powder metering and transport system and the aerosolisation system may be actuable through a coupled SMA wire assembly. For example, the SMA wire assembly may sequentially actuate metering, transport and aerosolisation.

Alternatively, as discussed supra, instead of a mechanism using SMA wires, the index wheel 558 may be driven by a gearing system linked to a rotary DC motor housed therein.

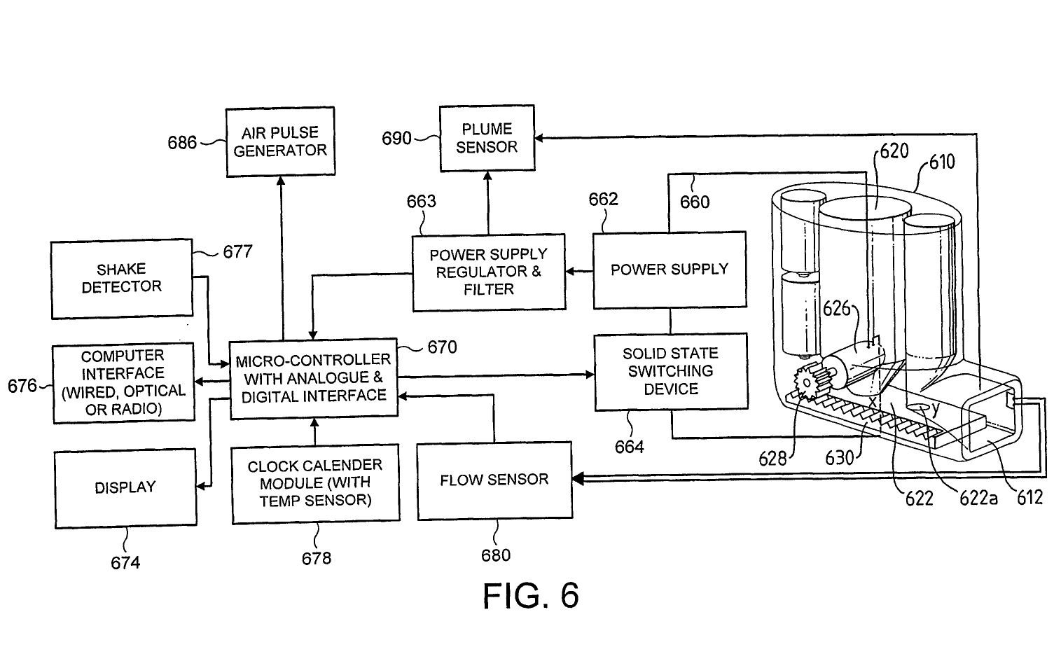

FIG. 6 shows a schematic representation of a breath-operable medicament dispensing system. The system comprises a metered dose inhaler similar to that shown in FIG. 3a comprising a tubular housing 610 having a dispensing outlet 612 in the form of a mouthpiece. Within the housing 610 sits a medicament reservoir which has a dose-metering mechanism (not shown). A slide plate 622 is transportable between a non-dispensing position X and a delivery position Y enabling the passage of dispensed dose in a medicament pocket 622a to the dispensing outlet 612.

A DC motor 626 drives a rotary gear wheel 628 which in turn drives the slide plate 622 by the rack 630. Control of electrical current flow to the DC motor 626 is achievable using the illustrated circuitry. The DC motor 626 is connected to actuation circuit 660 which includes a power supply 662 (e.g. a voltaic cell or battery of voltaic cells) and a switch 664 in the form of a solid state switching device. The switch 664 itself connects to control circuitry including microcontroller 670 which has an analogue and/or digital interface. The power supply for the control circuitry is taken from the power supply 662 after suitable regulation and filtering 663. The micro-controller 670 itself connects with a flow sensor 680 which is associated with a breath sensor 690.

It may be appreciated that current flow to the DC motor 626 and hence actuation of the transport means 622 may be achievable as follows. The patient inhales through the mouthpiece 624 resulting in a change in airflow within the housing 610. The change in airflow is detected by the flow sensor 680 which sends a signal to the micro-controller 670. The micro-controller 670, in turn sends a switching signal to the solid state switching device 664 which results in closing of the actuation circuit and electrical current flow therethrough. DC motor 626 thus drives the slide plate 622 from a non-dispensing position to a delivery position and hence, dispensing of the medicament to the inhaling patient.

It may also be seen in FIG. 6 that the micro-controller 670 is connected to a display 674 for display of information to the patient and also with a computer interface 676 for exchange of data therewith. Communication with the computer interface 676 may be via a wired, optical or radio communications link. The micro-controller 670 is may also be connected to shake detector 677 for use in detecting whether the container 620 is shaken prior to actuation of the transport means 622 and to a clock-calendar module 678 including a temperature sensor. All circuitry and components thereof including the power supply 662, display 674, shake detector 677, computer interface 676 and clock-calendar module 678 may be arranged to be present on the housing 610 such that the system is in the form of a discrete, hand-held device.

In addition, the micro-controller 670 is linked to an air pulse generator 686 for actuating the release mechanism for aerosolisation of the dose. The power supply 663 is connected to a plume sensor 690 which senses when a dose of medicament leaves the dispenser and feeds back to turn off the power supply.

It may be appreciated that any of the parts of the inhaler or actuator which contact the medicament suspension may be coated with materials such as fluoropolymer materials which reduce the tendency of medicament to adhere thereto. Any movable parts may also have coatings applied thereto which enhance their desired movement characteristics. Frictional coatings may therefore be applied to enhance frictional contact and lubricants used to reduce frictional contact as necessary.

It will be understood that the present disclosure is for the purpose of illustration only and the invention extends to modifications, variations and improvements thereto.

The application of which this description and claims form part may be used as a basis for priority in respect of any subsequent application. The claims of such subsequent application may be directed to any feature or combination of features described therein. They may take the form of product, method or use claims and may include, by way of example and without limitation, one or more of the following claims:

1. An inhaler device for delivery of a metered dose of dry powder medicament, the inhaler comprising a breath sensor for sensing the breath of a patient; means for transporting the medicament dose to a delivery position; and electro-mechanical coupling means for actuating said transport means to transport the medicament dose to said delivery position, wherein said coupling means is directly or indirectly responsive to the breath sensor.

2. An inhaler according to claim 1 wherein the medicament is pre-metered prior to actuation of the inhaler by the patient.

3. An inhaler according to claim 1 or claim 2 further comprising a reservoir for said dry powder and a meter for metering an amount of dry powder from said reservoir.

4. An inhaler according to claim 3 wherein the breath sensor actuates said meter.

5. An inhaler according to claim 4 wherein the coupling means is responsive to said meter.

6. An inhaler according to any one of the preceding claims further comprising release means.

7. An inhaler according to claim 6 wherein the release means is actuable by the coupling means and/or the meter and/or the transport means.

8. An inhaler according to any one of the preceding claims wherein the dose-release means comprises (i) a passive and/or (ii) an active dose-release mechanism.

9. An inhaler according to claim 8 wherein the passive dose-release mechanism comprises making the metered dose available to the patient for inhalation thereby.

10. An inhaler according to claim 8 or claim 9 wherein the active dose-release mechanism comprises means to propel pressurised gas in the direction of patient inhalation.

11. An inhaler according to claim 10 wherein the gas-propelling means provides at least one pulse of gas on actuation.

12. An inhaler according to claim 10 or 11 wherein the gas-propelling means provides one pulse of gas for each dose dispensed.

13. An inhaler according to any one of claims 10 to 12 wherein the gas is air.

14. An inhaler according to any one of claims 10 to 12 wherein the gas is an inert gas.

15. An inhaler according to any one of the preceding claims wherein the meter comprises a volume and/or a weight and/or a time and/or a surface-area and/or a particle counting regulated mechanism.

16. An inhaler according to any one of the preceding claims wherein the meter comprises a valve (for example, a linear or rotary valve) and/or a piston and/or a load cell and/or a plunger.

17. An inhaler according to any one of the preceding claims wherein the meter comprises at least one metering chamber.

18. An inhaler according to claim 17 wherein on actuation of the meter, the or each metering chamber moves into fluid communication with the reservoir.

19. An inhaler according to claim 17 or claim 18 wherein the meter and the reservoir are relatively rotatable with respect to each other about a common central axis.

20. An inhaler according to claim 19 wherein the or each metering chamber is adapted to be in fluid communication selectively with the reservoir or with the patient.

21. An inhaler according to any one of claims 17 to 20 wherein the or each metering chamber has a variable volume.

22. An inhaler according to any one of claims 17 to 20 wherein the or each metering chamber has a fixed volume which metering volume is variable by insertion of a plunger or piston.

23. An inhaler according to claim 21 wherein the or each metering chamber is formed from expandable material.

24. An inhaler according to claim 21 wherein the or each metering chamber has a telescopic or concertina arrangement.

25. An inhaler according to any one of claims 17 to 24 further comprising a gas permeable dry powder retaining means below the or each metering chamber.

26. An inhaler according to claim 25 wherein the retaining means is made from a gas-permeable filter, a mesh screen, a porous material or a perforated chamber element.

27. An inhaler according to any one of claims 6 to 26, additionally comprising a reset mechanism for resetting the meter and/or the transport means and/or the release means after actuation thereof.

28. An inhaler according to any one of the preceding claims additionally comprising climate control means.

29. An inhaler according to claim 28 wherein the climate control means is actuable by the coupling means and/or the transport means and/or the release means.

30. An inhaler according to claim 28 or claim 29 wherein the climate control means comprises means to (i) reduce moisture increase in the inhaler; and/or (ii) maintain ambient temperature; and/or (iii) dry the meter prior to actuation of the inhaler.

31. An inhaler according to any one of claims 28 to 30 wherein the climate control means comprises a desiccant.

32. An inhaler according to any one of claims 28 to 31 wherein the climate control means comprises a heater.

33. An inhaler according to claim 32 wherein the heater dries the meter prior to metering of the dose and/or immediately after the dose is dispensed.

34. An inhaler according to any one of claims 28 to 33 wherein the climate control means comprises a temperature and/or a moisture sensor.

35. An inhaler according to any one of the preceding claims wherein the coupling means comprises a spring and/or a lever.

36. An inhaler according to any one of the preceding claims wherein the coupling means comprises a solenoid.

37. An inhaler as claimed in any one of the preceding claims wherein the coupling means is reversibly deformable in response to heating thereof or application of a magnetic field thereto.

38. An inhaler according to claim 37, additionally comprising a reset coupling which is reversibly deformable in response to heating thereof or application of a magnetic field thereto.

39. An inhaler according to claims 37 or 38, wherein the heating is achievable by electric current flow through the coupling.

40. An inhaler according to any of claims 37 to 39, wherein the coupling comprises a wire, strip, coil or tube.

41. An inhaler according to claim 40, wherein the coupling comprises multiple wires, strips, coils or tubes.

42. An inhaler according to any of claims 37 to 41, wherein the coupling comprises one or more wires which contract in response to heating or application of a magnetic field thereto.

43. An inhaler according to claim 42, wherein the coupling exhibits a degree of contraction of from 2% to 8% on heating or application of a magnetic field thereto.

44. An inhaler according to claim 43, wherein the coupling comprises an alloy which undergoes a phase transition on heating or application of a magnetic field thereto.

45. An inhaler according to claim 44, wherein the alloy is a nickel-titanium alloy.

46. An inhaler according to claim 45, wherein said nickel-titanium alloy comprises from 5% to 95% nickel by weight and from 95% to 5% titanium by weight, preferably from 20% to 80% nickel by weight and from 80% to 20% titanium by weight.

47. An inhaler according to either of claims 45 or 46, wherein the nickel-titanium alloy additionally comprises copper, niobium or any mixtures thereof.

48. An inhaler according to claim 44, wherein the alloy is a copper-zinc-aluminium alloy or a copper-aluminium-nickel alloy.

49. An inhaler according to claim 44, wherein the alloy has the composition defined as Ni_{65-x-y}Mn₂₀+xGa₁₅+y, where x is between 3 atomic % and 15 atomic % and y is between 3 atomic % and 12 atomic %.

50. An inhaler according to claim 44, wherein the alloy has the composition defined as (NiₐFe_{b}Co_{c})_{65-x-y}(Mn_{d}FeₑCo_{f})₂₀+x(Ga_{g}SiₕAlᵢ)₁₅+y, where x is between 3 atomic % and 15 atomic % and y is between 3 atomic % and 12 atomic %, and where a+b+c=1, where d+e+f=1, and g+h+i=1.

51. An inhaler according to any of claims 40 to 50, wherein the one or more wires have a diameter from 30 to 400 micrometers, preferably from 50 to 150 micrometers.

52. An inhaler according to any of 40 to 51, wherein the coupling comprises from two to twelve, preferably six to ten wires which contract in response to heating or application of a magnetic field thereto.

53. An inhaler according to claim 40 or 41, wherein said strip comprises multiple layers of different metals.

54. An inhaler according to claim 53, wherein the strip comprises a bimetallic strip.

55. An inhaler according to either of claims 53 or 54, wherein the strip comprises at least one piezoelectric material.

56. An inhaler according to any of claims 37 to 55, wherein the coupling is deformable in response to heating arising from electrical current flow in the range from 0.01A to 100A, preferably from 0.1A to 5A.

57. An inhaler according to any of claims 37 to 55, wherein the coupling is deformable in response to a magnetic field of from 0.01 to 100 Tesla.

58. An inhaler according to any one of the preceding claims wherein the breath sensor electro-mechanically actuates the meter at a predetermined trigger point in the patient's breath cycle.

59. An inhaler according to claim 58 wherein the trigger point is during the inhalation or exhalation stage of the patient's breath cycle.

60. An inhaler according to any one of the preceding claims wherein the sensor comprises a breath-movable element which is movable in response to the breath of a patient.

61. An inhaler according to claim 60, wherein the breath-movable element is selected from the group consisting of a vane, a sail, a piston, a diaphragm and an impeller.

62. An inhaler according to any one of the preceding claims wherein the sensor comprises a pressure sensor for sensing the pressure profile associated with the breath of a patient.

63. An inhaler according to any one of the preceding claims wherein the sensor comprises an airflow sensor for sensing the airflow profile associated with the breath of a patient.

64. An inhaler according to any one of the preceding claims wherein the sensor comprises a temperature sensor for sensing the temperature profile associated with the breath of a patient.

65. An inhaler according to any one of the preceding claims wherein the sensor comprises a moisture sensor for sensing the moisture profile associated with the breath of a patient.

66. An inhaler according to any one of the preceding claims wherein the sensor comprises a gas sensor for sensing the chemical profile, for example, the oxygen or carbon dioxide profile associated with the breath of a patient.

67. An inhaler according to any one of the preceding claims wherein the sensor is connectable to an electronic information processor.

68. An inhaler according to claim 67 wherein the electronic information processor actuates the meter at a predetermined trigger point in the breath cycle.

69. An inhaler as claimed in claim 68 wherein the electronic information processor includes an active memory for storing information about the breath cycle.

70. An inhaler according to claim 68 wherein the electronic information processor includes a predictive algorithm for predicting the optimum trigger point.

71. An inhaler according to claim 68 wherein the electronic information processor includes a look up table for predicting the optimum trigger point.

72. An inhaler according to any one of claims 69 to 71 wherein the electronic information processor includes a second predictive algorithm for predicting the optimum amount of medicament to release.

73. An inhaler according to any one of claims 69 to 71 wherein the electronic information processor includes a second look up table for predicting the optimum amount of medicament to release.

74. An inhaler according to claim 69 to 73 wherein the electronic information processor includes a dose memory for storing information about earlier delivered doses and reference is made to the dose memory in predicting the optimum amount of medicament to release.

75. An inhaler according to claims 69 to 74 additionally comprising a display for displaying information about the optimum amount of medicament to release.

76. An inhaler according to any one of claims 67 to 75 additionally comprising a selector for selecting the amount of medicament to release.

77. An inhaler according to claim 76 wherein the selector is manually operable.

78. An inhaler according to claim 76 wherein the selector is operable in response to a signal from the electronic information processor.

79. An inhaler according to claim 76 to 78 wherein the selector comprises a timing mechanism for varying the time interval of activation of the inhaler.

80. An inhaler according to any one of claims 76 to 79 wherein the selector comprises a multiple-fire mechanism for multiple actuation of the inhaler wherein each actuation releases a portion of the optimum amount of medicament.

81. An inhaler according to any of the preceding claims, additionally comprising an electrical energy source.

82. An inhaler according to claim 81, wherein the electrical energy source comprises a voltaic cell or battery of voltaic cells.

83. An inhaler according to claim 81, wherein the voltaic cell or battery of voltaic cells is rechargeable.

84. An inhaler according to claim 81, wherein the electrical energy source comprises a photovoltaic cell or battery of photovoltaic cells.

85. An inhaler according to claim 81, wherein the electrical energy source comprises a converter for converting mechanical energy into electrical energy.

86. An inhaler according to any of claims 81 to 85, additionally comprising a controller for controlling the amount of electrical current flow through the coupling or to an electromagnet.

87. An inhaler according to any of claims 81 to 86, additionally comprising a timer for controlling the duration of electrical current flow through the coupling or to an electromagnet.

88. An inhaler according to any of claims 81 to 87 additionally comprising a local electrical energy store.

89. An inhaler according to any one of claims 81 to 88 wherein the additional energy source is mechanically-generated.

90. An inhaler according to claim 89 wherein the energy source comprises a biasable resilient member.

91. An inhaler according to claim 90 wherein the biasable resilient member is a spring.

92. An inhaler according to claim 89 wherein the energy source comprises a source of compressed fluid, preferably compressed gas.

93. An inhaler according to claim 89 wherein the energy source comprises a chemical energy store, preferably a chemical propellant or ignition mixture.

94. An inhaler according to claim 89 wherein the energy source comprises a physically explosive energy source.

95. An inhaler according to any one of the preceding claims wherein the medicament is selected from the group consisting of albuterol, salmeterol, fluticasone propionate, beclomethasone dipropionate, salts or solvates thereof and any mixtures thereof.

96. An inhaler according to any one of the preceding claims wherein the dry powder medicament includes a pharmaceutical excipient in dry powder form.

97. An inhaler according to any one of the preceding claims wherein the density of the dry powder medicament particles is reduced relative to standard dry powder medicament.

98. An inhaler according to any one of the preceding claims wherein the dry powder medicament particles are aerodynamically shaped to improve medicament delivery to the patient.

99. An inhaler according to any one of the preceding claims comprising an actuation counter for counting the number of actuations of the meter and/or transport means and/or dose-release means or a dose counter for counting the number of doses delivered.

100. An inhaler according to claim 99, wherein the actuation counter is independent of the coupling between the breath sensor and the transport means and optionally dose-release means.

101. An inhaler according to any one of the preceding claims additionally comprising a safety mechanism to prevent unintended multiple actuations of the inhaler.

102. An inhaler according to claim wherein the safety mechanism imposes a time delay between successive actuation of the inhaler.

103. An inhaler according to any of the preceding claims comprising a manual override enabling manual actuation of the transport means.

104. An inhaler according to claim 103 comprising a child resistance feature to prevent undesirable actuation thereof by children.

105. An actuator for use in an inhaler according to any one of the preceding claims.

106. An actuator for a dry powder medicament container having a meter, the actuator comprising a dispenser seat for receipt of the meter, a breath sensor, and transport means, wherein the transport means is electro-mechanically actuable by the breath sensor and/or the meter.

107. An actuator according to claim 106 further comprising release means.

108. An actuator according to claim 106 or 107 wherein the breath sensor is linkable to the transport means and/or dose-release means via coupling means.

109. An actuator according to claim 108 wherein the coupling means is reversibly deformable in response to heating thereof or application of a magnetic field thereto.

110. Kit of parts comprising an inhaler according to any of claims 1 to 104 in the form of a cartridge; and a housing shaped for receipt of the cartridge.

111. A method for the delivery of a metered dose of dry powder medicament to a patient comprising:
(i) sensing the breath of a patient by use of a breath sensor;
(ii) transporting the medicament dose to a delivery position by use of transport means; and optionally
(iv) releasing the medicament dose for inhalation by the patient by the use of dose-release means,
wherein electro-mechanical coupling means actuate said transport means and optionally said release means, and said coupling means is directly or indirectly responsive to the breath sensor.

112. A method as claimed in claim 111 wherein the medicament is pre-metered prior to actuation of the inhaler by the patient.

113. A method according to claim 111 or claim 112 further comprising metering a volume of dry powder from a medicament reservoir after sensing the breath of a patient and prior to transporting the medicament to a delivery position.

114. A method according to claim 113 wherein the metering step is actuable by the breath sensor.
